(19) United States
(12) Patent Application Publication
(10) Pub. No.: US 2007/0071807 A1
(43) Pub. Date: Mar. 29, 2007

(54) CAPSULE-TYPE DRUG-RELEASING DEVICE AND CAPSULE-TYPE DRUG-RELEASING DEVICE SYSTEM

(76) Inventors: Hidero Akiyama, Shibuya-ku (JP); Mizuo Nakayama, Shibuya-ku (JP); Takehiko Matsumura, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/536,194

(22) Filed: Sep. 28, 2006

Related U.S. Application Data
(60) Provisional application No. 60/728,635, filed on Oct. 20, 2005.

(30) Foreign Application Priority Data
Sep. 28, 2005 (JP) 2005-281138

(51) Int. Cl. A61N 1/00; A61K 9/48

(52) U.S. Cl. 424/451; 607/2

(57) ABSTRACT

A capsule-type drug-releasing device is capable of freely controlling the release and stoppage of a drug and causing neither the back flow of contents of a stomach, an intestine, or the like nor clogging with the contents. A capsule-type drug-releasing device may include: a capsule-shaped casing; a cation exchange membrane arranged on the surface of the casing; a drug solution holding portion arranged inside the casing and on the inner side of the cation exchange membrane; an anion exchange membrane arranged on the inner side of the drug solution holding portion; an electrolyte solution holding portion arranged on the inner side of the anion exchange membrane; an electrode arranged on the inner side of the electrolyte solution holding portion; an electric power source connected to the electrode; a control portion capable of controlling energization from the electric power source to the electrode; and a transceiver portion causing the control portion to receive a signal from the outside of an organism.

### CROSS-REFERENCE TO RELATED APPLICATION

This application claims the benefit under 35 U.S.C. § 119(e) of U.S. Provisional Patent Application No.60/728,635, filed Oct. 20, 2005.

### BACKGROUND

### Field

The present disclosure relates to a capsule-type drug-releasing device for releasing a drug at a target site in a body cavity such as a stomach or an intestine.

### Description

The following procedure has been conventionally performed. That is, a drug is sealed in a capsule, and one swallows the capsule, with the result that the drug is released at a target site in a body cavity such as a stomach or an intestine. For example, a capsule for drug administration described in Japanese patent application JP2005-102851A and a capsule-type medical device described in Japanese patent application JP2003-325438A have been known.

FIG. 8 shows the capsule for drug administration described in Patent Document 1. A capsule 1 for drug administration includes a reservoir 5 for holding a drug 2, and the reservoir 5 includes a heater 4. In addition, multiple drug ports 3 are arranged in part of a capsule-like casing. A thin film is formed in each of the drug ports 3 in such a manner that the drug 2 does not leak out. After one has swallowed the capsule 1 for drug administration, at the time when the capsule 1 for drug administration reaches a desired position, the heater 4 is caused to generate heat by a signal transmitted from the outside (e.g., the outside of a body). The heat generated by the heater 4 causes the drug 2 in the reservoir 5 to thermally expand, and, furthermore, to boil. A pressure generated as a result of the expansion and boiling causes the drug 2 to break through the thin films provided to the drug ports 3. As a result, the drug 2 can be released to a desired position in a body cavity.

On the other hand, FIGS. 9A and 9B each show a capsule-type medical device 11 described in Patent Document 2.

The capsule-type medical device 11 includes a storage chamber 15 storing a drug 12, and the storage chamber 15 has an opening 13 communicating with the outside. The path of the opening 13 is blocked by a movable body 14. The movable body 14 includes a permanent magnet 17. Furthermore, the movable body 14 includes a screw portion. In addition, part of the movable body 14 includes a hole 16. The right side of FIG. 9A shows, for example, an image capture portion for imaging the inside of a body cavity.

The permanent magnet 17 can be rotated by a controlled magnetic force from the outside (e.g., the outside of a body). That is, at the stage where one swallows the capsule-type medical device 11, the movable body 14 is positioned in such a manner that the hole 16 provided to the movable body 14 is formed at a position shifted from the opening 13 of the storage chamber 15. However, when the swallowed capsule-type medical device 11 is placed at a desired position (e.g., an affected site of a stomach, an intestine, or the like), the controlled magnetic force from the outside causes the permanent magnet 17 to rotate. The rotation causes the movable body 14 to move toward the right side of each of FIGS. 9A and 9B. As a result, as shown in FIG. 9B, the opening 13 of the storage chamber 15 and the hole 16 communicate with each other. The communication causes the drug 12 in the storage chamber 15 to be released to the outside of the device inside of a body.

However, in the case of the capsule 1 for drug administration described in JP2005-102851A, when once the heater 4 is actuated to cause the drug 2 in the reservoir 5 to break through the thin films provided to the drug ports 3, the drug 2 is continuously released. It is impossible to stop the release of the drug 2. That is, the capsule 1 for drug administration is not useful if specific sites to which one desires to release a drug are present at multiple places distant from each other in a body cavity such as a stomach or an intestine and he or she desires to release the drug only in the vicinities of the specific sites.

In addition, in the capsule-type medical device 11 described in JP2003-325438A, for example, controlled magnetism causes the opening 13 starting from the storage chamber 15 and the hole 16 provided to the movable body 14 to communicate with each other, or the communication is blocked, with the result that the release and stoppage of the drug 12 can be controlled. However, it is complicated to provide a capsule sufficiently small enough to be swallowed with a mechanism with which such control can be attained. Further, the amount of a drug that can be stored is also limited. Furthermore, a stomach, intestine, or the like contains a large number of contents produced as a result of the digestion of food. Such content may flow back into the storage chamber 15 through the hole 16 and the opening 13 communicating with each other. In addition, part of the hole 16 or the opening 13 may be clogged with the contents.

### SUMMARY

The present disclosure addresses at least some of these problems. One embodiment may be summarized as a capsule-type drug-releasing device capable of: freely controlling the release and stoppage of a drug with the aid of control from the outside (the outside of a body); and surely releasing the drug to the outside of a capsule (the inside of an organism) without any structural change involved in the release and stoppage of the drug, and a system using the drug-releasing device.

The embodiment may be summarized as a capsule-type drug release apparatus that includes at least: a housing in a shape of a capsule; a first ion exchange member capable of selectively passing an ion of a first polarity therethrough, the first ion exchange member being disposed on a surface of the housing in the shape of a capsule; a drug solution holding portion disposed in the housing and inside the first ion exchange member for holding a drug ion of the first polarity; a second ion exchange member disposed further inside the drug solution holding portion, the second ion exchange member being capable of selectively passing an ion of a second polarity therethrough; an electrolyte solution holding portion disposed further inside the second ion exchange member; electrodes disposed further inside the electrolyte solution holding portion; an electric power source connected to the electrodes; a control portion capable of controlling energization from the electric power source to the electrodes; and a receiving portion where the control portion receives a signal from the outside of an organism.

Such an embodiment may permit control over the release and stoppage of a drug. Upon release of the drug, no structural motions such as the opening and closing of a hole through which the outside of a capsule and the inside of the capsule communicate with each other occur. The contents of a stomach, intestine, or the like may be advantageously prevented from flowing back into the portion of the hole, and the portion of the hole is not clogged with the contents.

Each of the terms “ion of a first polarity” and “ion of a second polarity” as used herein refers to either a cation or an anion. For example, when the “ion of the first polarity” refers to a cation, the “ion of the second polarity” refers to an anion.

In addition, the ion exchange members may be formed of an ion exchange membrane.

As a result, various commercially available ion exchange membranes can be used, so production cost can be reduced.

Furthermore, the capsule-type drug release apparatus may include a transmitting portion capable of transmitting a signal from the control portion to the outside of the organism.

As a result, data (such as the amount of a drug to be released and a time period for releasing the drug) can be acquired from the drug-releasing device. The release of the drug can be controlled by means of the data.

As shown in embodiments described below, the capsule-type drug release apparatus system may include: a capsule-type drug release apparatus including at least a housing in a shape of a capsule, a first ion exchange member capable of selectively passing an ion of a first polarity therethrough, the first ion exchange member being disposed on a surface of the housing in the shape of a capsule, a drug solution holding portion disposed in the housing and inside the first ion exchange member for holding a drug ion of the first polarity, a second ion exchange member disposed further inside the drug solution holding portion, the second ion exchange member being capable of selectively passing an ion of a second polarity therethrough, an electrolyte solution holding portion disposed further inside the second ion exchange member, electrodes disposed further inside the electrolyte solution holding portion, an electric power source connected to the electrodes, a control portion capable of controlling energization from the electric power source to the electrodes, and a receiving portion where the control portion receives a signal from the outside of an organism; position determining means capable of determining a position of the capsule-type drug release apparatus in the organism; and transmitting means capable of transmitting a signal from the outside of the organism, the signal being received by the receiving portion.

As a result, a drug sealed in a capsule can be accurately released at a site (specific site) in a body cavity such as a stomach or intestine where it is necessary or desirable for the drug to be released while the position of the drug-releasing device in an organism is identified.

The capsule-type drug release apparatus system may include the capsule-type drug release apparatus that further includes a transmitting portion capable of transmitting a signal from the control portion to the outside of the organism, and the capsule-type drug release apparatus system that further includes receiving means capable of receiving a signal outside the organism, the signal being transmitted by the transmitting portion.

As a result, the amount of a drug to be released and a time period for releasing the drug are acquired, with the result that the excessive release or the like of the drug can be prevented, and a side effect or the like due to the drug can be reduced.

The application of the present disclosure enables a capsule-type drug-releasing device to freely control the release and stoppage of a drug. In addition, no mechanical structural motions are involved in the release and stoppage of the drug occur, so contents of a stomach, intestine, or the like may not flow back, and an opening may not be clogged with the contents.

Hereinafter, an example of an embodiment of the present invention will be described in detail with reference to the accompanying drawings.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram of a drug-releasing device according to one illustrated embodiment.

FIG. 2 is a cross-sectional diagram taken along the line II-II of FIG. 1.

FIG. 3 is a schematic diagram showing the movement of the drug-releasing device in a body according to one illustrated embodiment.

FIG. 4 is a sectional diagram of an intestine.

FIG. 5 is a schematic diagram of a drug-releasing device according to another illustrated embodiment.

FIG. 6 is a cross-sectional diagram taken along the line VI-VI in FIG. 5.

FIG. 7 is a schematic diagram showing an example in which a drug-releasing device is surrounded by a chitosan capsule according to one illustrated embodiment.

FIG. 8 is a cross-sectional view of a capsule for drug administration described in Patent Document 1.

FIGS. 9A and 9B are sectional diagrams each showing a conventional capsule-type medical device. FIG. 9A shows a state before a drug is released, and FIG. 9B shows a state at the time of the release of the drug.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with medical devices, for example, iontophoresis devices and/or regulators or controllers have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

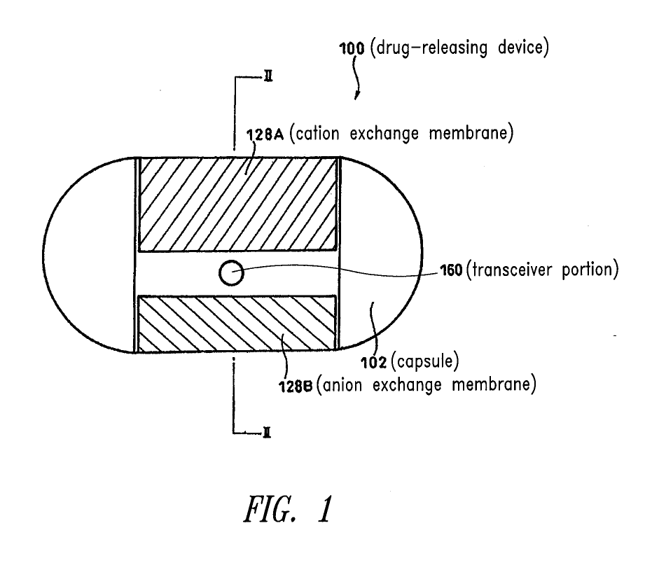

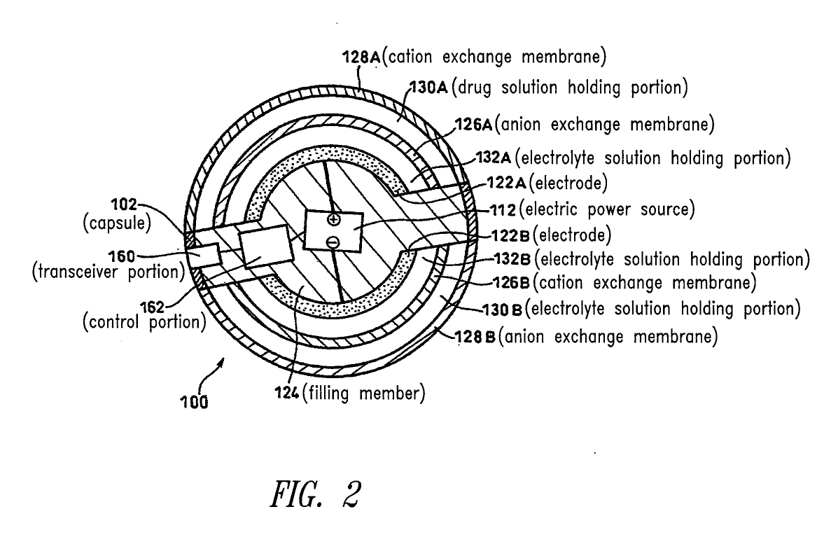

FIG. 1 is a schematic view of a drug-releasing device 100 according to one illustrated embodiment of the present disclosure. FIG. 2 is a sectional view taken along the line II-II in FIG. 1.

The drug-releasing device 100 has a so-called capsule-type shape obtained by providing both ends of a cylinder with hemispherical bodies. In addition, a cation exchange membrane 128A capable of selectively passing a cation (an ion of the first polarity) and an anion exchange membrane 128B capable of selectively passing an anion (an ion of the second polarity) are present on the outer peripheral surface (surface) of the cylindrical portion of a capsule 102 as a casing. In addition, a transceiver portion 160 for at least one of transmitting/receiving a signal to/from the outside of the capsule and the outside of an organism when the device is used is arranged on part of the outer peripheral surface of the capsule 102, the part being placed between the cation exchange membrane 128A and the anion exchange membrane 128B. In addition, an appropriate seal material (not shown) is interposed between the capsule 102 and each of the cation exchange membrane 128A, the anion exchange membrane 128B, and the transceiver portion 160 present on the outer peripheral surface of the capsule to strictly prevent, for example, the penetration of a digestive juice or the like into the capsule.

Next, description will be made with reference to FIG. 2.

A drug solution holding portion 130A is arranged inside the capsule 102 and on the inner side of the cation exchange membrane 128A. The term “inner side” as used herein refers to a side close to the center of a capsule-like drug-releasing device. Hereinafter, such side is simply referred to as the “inner side”. Furthermore, an anion exchange membrane 126A is arranged on the inner side of the drug solution holding portion 130A. Furthermore, an electrolyte solution holding portion 132A is arranged on the inner side of the anion exchange membrane 126A. Furthermore, an electrode 122A is arranged on the inner side of the electrolyte solution holding portion 132A. The electrode is connected to the anode of an electric power source 112 placed at a substantially central portion of the drug-releasing device 100.

Meanwhile, the anion exchange membrane 128B is present on a side at an angle of 180° with respect to the cation exchange membrane 128A in the capsule 102. An electrolyte solution holding portion 130B is arranged inside the capsule 102 and on the inner side of the anion exchange membrane 128B. Furthermore, a cation exchange membrane 126B is arranged on the inner side of the electrolyte solution holding portion 130B. Furthermore, an electrolyte solution holding portion 132B is arranged on the inner side of the cation exchange membrane 126B. Furthermore, an electrode 122B is arranged on the inner side of the electrolyte solution holding portion 132B. The electrode 122B is connected to the cathode of the electric power source 112. The two electrolyte solution holding portions 130B and 132B are not necessarily separated into two portions through an ion exchange membrane, and may be constituted integrally.

A control portion 162 controls energization/non-energization from the electric power source 112 to the electrodes 122A and 122B. Furthermore, the control portion 162 is connected to the transceiver portion 160 capable of receiving a signal from the outside of an organism and of transmitting a signal to the outside of the organism.

For convenience, description will be made by taking, as an example, a drug-releasing device for administering a drug whose drug component dissociates to cations. In contrast, a drug-releasing device for administering a drug whose drug component dissociates to anions can be constituted by switching the polarity (i.e., plus or minus) of each of: a voltage applied to an electrode; and an ion exchange membrane in the above-described constitution.

The capsule 102 as a casing is constituted by a material which is inactive or inert even in a body cavity such as a stomach or an intestine. Furthermore, the inside of the capsule 102 is filled with a filling member 124. The filling member 124 is sealed in such a manner that the capsule 102 and each member to be described later are brought into contact with each other. As a result, the penetration of a digestive juice or the like from a gap is prevented even when one swallows the capsule 102 in his or her body. The entirety of the inside of the capsule is not necessarily filled with the filling member, and a space may be present in the capsule as long as sufficient sealing property can be secured. Placing a space may advantageously reduce the weight of the device.

Next, any variety of conductive materials can be used for each of the electrodes 122A and 122B. When the electrolyte solution holding portions 132A and 132B to be described later are present like this embodiment, a carbon electrode may be preferred.

Subsequently, the electrolyte solution holding portions 130B, 132A, and 132B are each intended for holding an electrolyte solution for securing the energization property of the drug-releasing device for a long time period. A phosphate buffer saline or an aqueous solution of an organic acid can be used as the electrolyte solution. An electrolyte that is more likely to be oxidized or reduced than the electrolytic reaction of water (i.e., an oxidation reaction at an anode electrode and a reduction reaction at a cathode electrode) such as: an inorganic compound such as ferrous phosphate or ferric phosphate; a substance such as ascorbic acid (vitamin C) or sodium ascorbate; an organic acid such as lactic acid, oxalic acid, malic acid, succinic acid, or fumaric acid and/or a salt of the organic acid; or a mixture of them is more preferably used. In this case, the generation of a gas due to the electrolysis of water, and an increase in conductive resistance or a fluctuation in pH value due to the generation can be prevented. Of course, an electrolyte is not construed as being limited to the substances described here.

Next, the cation exchange membrane 128A having a function of selectively passing a cation, and, for example, a cation exchange membrane such as a NEOSEPTA (CM-1, CM-2, CMX, CMS, or CMB) manufactured by Tokuyama Co., Ltd can be used for the cation exchange membranes without any particular limitation. In addition, a cation exchange membrane of a type in which a porous film composed of a polyolefin resin, a vinyl chloride-based resin, a fluorine-based resin, a polyamide resin, a polyimide resin, or the like having cavities a part or whole of which are filled with a cation exchange resin can be particularly preferably used. In this case, the pores can be filled with the cation exchange resin by: impregnating the cavities of the porous film with a solution prepared by blending a crosslinkable monomer such as styrene-divinylbenzene or chloromethylstyrene-divinylbenzene with a polymerization initiator; polymerizing the resultant; and introducing, into the polymer, a cation exchange group such as a sulfonic group, a carboxylic group, or a phosphoric group.

Next, the anion exchange membrane 126A is an ion exchange membrane having a function of selectively passing an anion, and, for example, an anion exchange membrane such as a NEOSEPTA (AM-1, AM-3, AMX, AHA, ACH, or ACS) manufactured by Tokuyama Co., Ltd can be used for the anion exchange membranes without any particular limitation. In addition, an anion exchange membrane of a type in which a porous film composed of a polyolefin resin, a vinyl chloride-based resin, a fluorine-based resin, a polyamide resin, a polyimide resin, or the like having cavities a part or whole of which are filled with an anion exchange resin may be particularly preferred. In this case, the pores can be filled with the anion exchange resin by: impregnating the cavities of the porous film with a solution prepared by blending a crosslinkable monomer such as styrene-divinylbenzene or chloromethylstyrene-divinylbenzene with a polymerization initiator; polymerizing the resultant; and introducing, into the polymer, an anion exchange group such as a primary amino group, a secondary amino group, a tertiary amino group, a quaternary ammonium group, a pyridyl group, an imidazole group, a quaternary pyridinium group, or a quaternary imidazolium group.

Next, the drug solution holding portion 130A holds a solution (drug ion) of a drug whose drug component dissociates to cations as a result of dissolution.

Next, the action of the drug-releasing device 100 will be described.

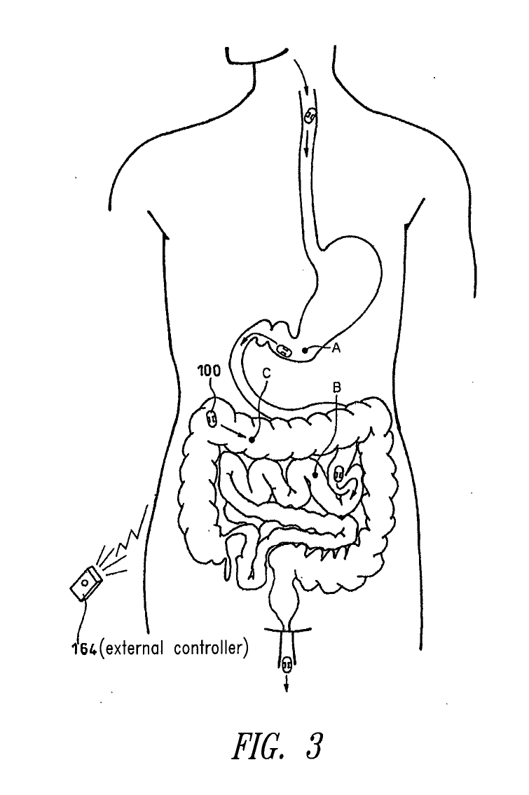

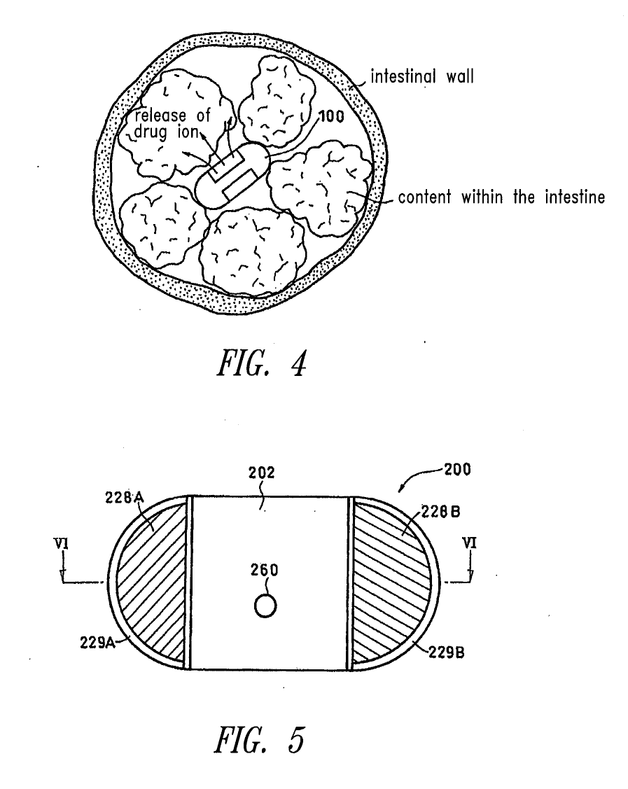

As shown in FIG. 3, when a patient swallows the drug-releasing device 100, the device is discharged to the outside of the body through a stomach, a small intestine, and a large intestine sequentially by virtue of, for example, a peristaltic movement. At this time, suppose that one desires to release a drug to multiple specific sites (e.g., a specific site A, a specific site B, and a specific site C). The position of the drug-releasing device 100 swallowed by the patient in his or her body is always or periodically identified by capsule position identifying means using, for example, X-rays. When the drug-releasing device 100 reaches a specific site, a signal is transmitted from an external controller 164 on the basis of the position identified by the capsule position identifying means. The external controller 164 is transmitting means capable of transmitting a signal to the transceiver portion 160 enabling the control portion 162 provided to the drug-releasing device 100 to receive a signal from the outside of an organism. At the same time, the controller functions as receiving means for receiving a signal from the transceiver portion 160 that transmits a signal from the control portion 162 to the outside of the organism. Upon reception of the signal from the external controller 164, the control portion 162 starts or stops energization from the electric power source 112 to the electrodes 122A and 122B. When the current from the electric power source 112 propagates to the electrode 122A, the electrolyte solution in the electrolyte solution holding portion 132A is oxidized. For example, ferrous phosphate is changed to ferric phosphate. In this case, a balance between a cation and an anion in the electrolyte solution holding portion 132A is lost (the amount of cations increases). A cation of the electrolyte solution holding portion 132A starts to move toward the drug solution holding portion 130A in order to compensate for the balance. On the other hand, an anion of the drug solution holding portion 130A starts to move toward the electrolyte solution holding portion 132A. However, owing to the presence of the anion exchange membrane 126A placed between the electrolyte solution holding portion 132A and the drug solution holding portion 130A, a cation cannot pass, and only an anion is selectively passed. That is, no movement of a cation from the electrolyte solution holding portion 132A to the drug solution holding portion 130A is permitted, and only the movement of an anion from the drug solution holding portion 130A to the electrolyte solution holding portion 132A is permitted. In this case, a balance between a cation and an anion in the drug solution holding portion 130A is lost. A cation of the drug solution holding portion 130A starts to move toward the outside of the drug-releasing device 100 (the inside of a body) through the cation exchange membrane 128A in order to compensate for the balance. At this time, a cation is selectively passed by the cation exchange membrane 128A, so it can move (be released) to the outside of the drug-releasing device 100.

When the current from the electric power source 112 propagates to the electrode 122B, the electrolyte solution in the electrolyte solution holding portion 132B is reduced. For example, ferric phosphate is changed to ferrous phosphate. In this case, a balance between a cation and an anion in the electrolyte solution holding portion 132B is lost (the amount of anions increases). An anion of the electrolyte solution holding portion 132B starts to move toward the electrolyte solution holding portion 130B in order to compensate for the balance. On the other hand, a cation of the electrolyte solution holding portion 130B starts to move toward the electrolyte solution holding portion 132B. However, owing to the presence of the cation exchange membrane 126A placed between the electrolyte solution holding portion 132A and the electrolyte solution holding portion 130B, an anion cannot pass, and only a cation is selectively passed. That is, no movement of an anion from the electrolyte solution holding portion 132B to the electrolyte solution holding portion 130B is permitted, and only the movement of a cation from the electrolyte solution holding portion 130B to the electrolyte solution holding portion 132B is permitted. In this case, a balance between a cation and an anion in the drug solution holding portion 130B is lost. An anion of the electrolyte solution holding portion 130B starts to move toward the outside of the drug-releasing device 100 through the anion exchange membrane 128B in order to compensate for the balance. At this time, an anion is selectively passed by the anion exchange membrane 128B, so it can move to the outside of the drug-releasing device 100.

Through such action, as shown in FIG. 4, the drug-releasing device 100 can release the drug at a desired specific site.

In contrast to the above description, when no current flows from the electric power source 112 to the electrodes 112A and 112B, no ion movement occurs, so the release of the drug immediately stops.

As described above, the drug-releasing device 100 is adapted to be capable of freely controlling the release or stoppage of a drug only by virtue of an electrical action without any structural change involved in the release/stoppage of the drug. In addition, similarly, no time lag resulting from a mechanical structural motion is present, so the release/stoppage of the drug can be controlled in an improved linear fashion.

In this embodiment, even when electricity is supplied from the electric power source 112 by the actions of the electrolyte solution holding portions 132A and 132B adjacent to the electrodes 122A and 122B, and, furthermore, of the electrolyte solution having a buffer action present in each of the electrolyte solution holding portions 132A and 132B, a gas such as chlorine or hydrogen is not generated, and there is no risk of the explosion or the like of the drug-releasing device 100 involved in the generation of the gas. In addition, a pressure sensitive device (such as a pressure sensor) may be added if required.

In addition, the control portion 162 can transmit data (such as a time period for which the electric power source 112 is turned on and the amount of a drug to be released) to the outside of an organism via the transceiver portion 160. The above-described external controller 164 receives and manages the data, with the result that the amount of a drug necessary for each specific site or the like can be controlled, and a side effect or the like occurring as a result of the release of a drug in an amount more than necessary can be reduced.

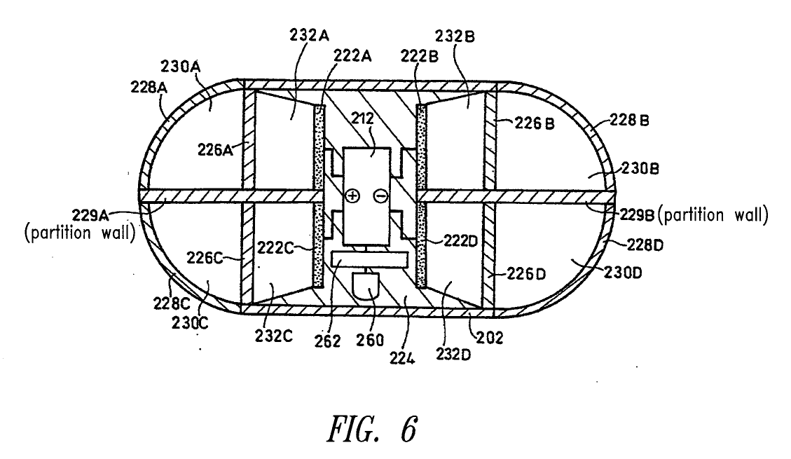

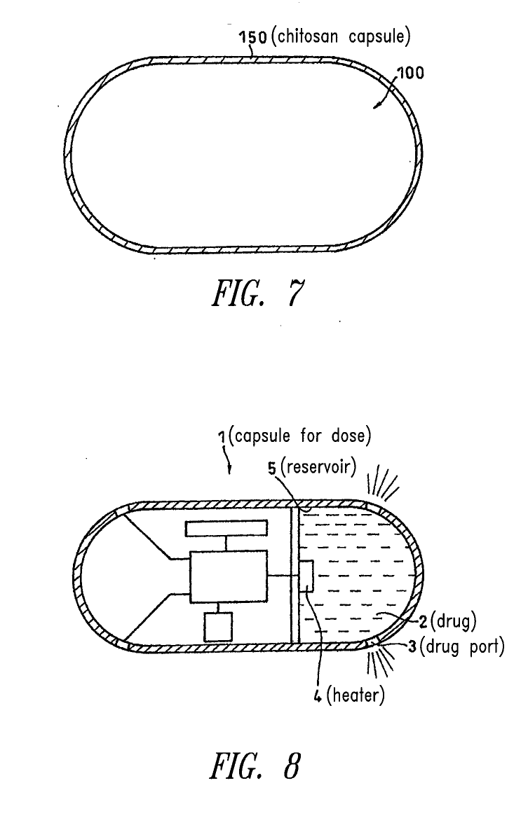

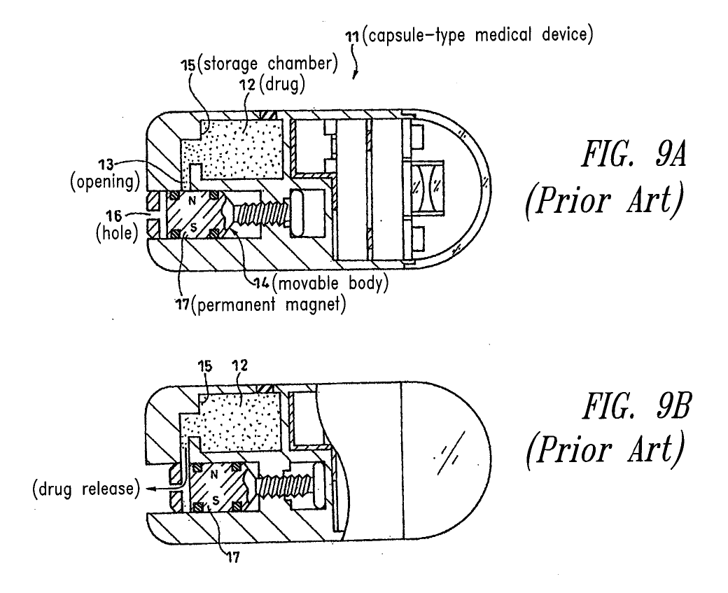

Subsequently, an example of another illustrated embodiment will be described with reference to FIGS. 5 and 6.

FIG. 5 is a general view of a drug-releasing device 200. FIG. 6 is a sectional view taken along the line VI-VI in FIG. 5. Portions identical or similar to those of the above-described drug-releasing device 100 are merely provided with reference numbers each having the same two least significant digits as those of each of the reference numbers of the device 100, and duplicate description of a constitution and an action is omitted.

The drug-releasing device 200 is mainly characterized in that it has multiple drug solution holding portions. That is, when one desires to release different drugs to multiple specific sites, the respective drug solution holding portions hold the different drugs, with the result that each drug can be released at a timing unique to the drug.

In a capsule-like casing 202 of the drug-releasing device 200, an anion exchange membrane is arranged in one hemispherical portion, and a cation exchange membrane is arranged in the other hemispherical portion. Two cation exchange membranes 228A and 228C through the intermediation of a partition plate 229A are arranged in one hemispherical portion. Drug solution holding portions 230A and 230C holding different drugs are placed on the inner sides of the cation exchange membranes 228A and 228C, respectively. Furthermore, anion exchange membranes 226A and 226C are placed on the inner sides of the drug solution holding portions 230A and 230C, respectively. Furthermore, electrolyte solution holding portions 232A and 232C are placed on the inner sides of the anion exchange membranes 226A and 226C, respectively. Furthermore, electrodes 222A and 222C are placed on the inner sides of the electrolyte solution holding portions 232A and 232C, respectively. The two electrodes 222A and 222C are both connected to the anode of an electric power source 212.

Meanwhile, two anion exchange membranes 228B and 228D through the intermediation of a partition plate 229B are arranged in the hemispherical portion opposite to the hemispherical portion having the cation exchange membranes 228A and 228C on its surface. Furthermore, electrolyte solution holding portions 230B and 230D are placed on the inner sides of the anion exchange membranes 228B and 228D, respectively. Furthermore, cation exchange membranes 226B and 226D are placed on the inner sides of the electrolyte solution holding portions 230B and 230D, respectively. Furthermore, electrolyte solution holding portions 232B and 232D are placed on the inner sides of the cation exchange membranes 226B and 226D, respectively. Furthermore, electrodes 222B and 222D are placed on the inner sides of the electrolyte solution holding portions 232B and 232D, respectively. The two electrodes 222B and 222D are both connected to the cathode of the electric power source 212.

When the two drug solution holding portions 230A and 230C hold different drugs in such constitution, in the case where the drug-releasing device 200 moving in a body is located, for example, in a stomach, a peptogenic agent can be released at the specific site A, and, in the case where the device 200 is located at a large intestine portion, a dejection promoter or the like can be released at the specific site C.

As in the case of the drug-releasing device 100 described at first, the drug solution holding portions 230A and 230C are each caused to hold a drug, and, at the same time, the electrolyte solution holding portions 230B and 230D connected to the cathode side of the electric power source 212 are each caused to hold a drug whose drug component dissociates to anions, with the result that the drug-releasing device 200 can hold and release a maximum of four kinds of drugs (e.g., a maximum of two kinds of drugs in the case of the above-described drug-releasing device 100).

Of course, the device can be allowed to hold and release an increased number of drugs by partitioning drug solution holding portions by means of a partition plate.

In addition, as shown in FIG. 7, the entirety of a drug-releasing device may be surrounded by a chitosan capsule 150 or the like before it is administered to a body. With such constitution, the device can move to a small intestine without being digested by pepsin in a stomach owing to the chitosan capsule 150, and the chitosan capsule is decomposed by bacteria present in the small intestine or the like, with the result that a drug can be released. In this case, each ion exchange membrane present on the surface of the drug-releasing device can be protected from the digestive juice of the stomach.

The present invention is applicable to not only a drug-releasing device to be used for a human body but also a drug-releasing device used for a wide variety of animals.

Description of Reference Numerals

| | |
|---|---|
| 100 | drug-releasing device |
| 102 | capsule |
| 112 | electric power source |
| 122A, 122B | electrode |
| 124 | filling member |
| 126A, 128B | anion exchange membrane |
| 126B, 128A | cation exchange membrane |
| 130A | drug solution holding portion |
| 130B, 132A, 132B | electrolyte solution holding portion |
| 150 | chitosan capsule |
| 160 | transceiver portion |
| 162 | control portion |
| 164 | external controller |

All of the above U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 60/728,635, filed Oct. 20, 2005, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We/I claim:

1. A capsule-type drug release apparatus, comprising:
a housing in a shape of a capsule;
a first ion exchange member capable of selectively passing an ion of a first polarity therethrough, the first ion exchange member being disposed on a surface of the housing in the shape of a capsule;
a drug solution holding portion disposed in the housing and inside the first ion exchange member for holding a drug ion of the first polarity;
a second ion exchange member disposed further inside the drug solution holding portion, the second ion exchange member being capable of selectively passing an ion of a second polarity therethrough, the second polarity different from the first polarity;
an electrolyte solution holding portion disposed further inside the second ion exchange member;
electrodes disposed further inside the electrolyte solution holding portion;
an electric power source connected to the electrodes;
a control portion capable of controlling energization/disenergization from the electric power source to the electrodes; and
a receiving portion where the control portion receives a signal from the outside of an organism.

2. The capsule-type drug release apparatus according to claim 1, further comprising a transmitting portion capable of transmitting a signal from the control portion to the outside of the organism.

3. The capsule-type drug release apparatus according to claim 1, wherein each of the ion exchange members is formed of an ion exchange membrane.

4. The capsule-type drug release apparatus according to claim 3, further comprising a transmitting portion capable of transmitting a signal from the control portion to the outside of the organism.

5. A capsule-type drug release apparatus system, characterized by comprising:
a capsule-type drug release apparatus comprising at least
a housing in a shape of a capsule,
a first ion exchange member capable of selectively passing an ion of a first polarity therethrough, the first ion exchange member being disposed on a surface of the housing in the shape of a capsule,
a drug solution holding portion disposed in the housing and inside the first ion exchange member for holding a drug ion of the first polarity,
a second ion exchange member disposed further inside the drug solution holding portion, the second ion exchange member being capable of selectively passing an ion of a second polarity therethrough,
an electrolyte solution holding portion disposed further inside the second ion exchange member,
electrodes disposed further inside the electrolyte solution holding portion,
an electric power source connected to the electrodes,
a control portion capable of controlling energization from the electric power source to the electrodes, and
a receiving portion where the control portion receives a signal from the outside of an organism;
position determining means capable of determining a position of the capsule-type drug release apparatus in the organism; and
transmitting means capable of transmitting a signal from the outside of the organism, the signal being received by the receiving portion.

6. The capsule-type drug release apparatus system according to claim 5 wherein
the capsule-type drug release apparatus further comprises a transmitting portion capable of transmitting a signal from the control portion to the outside of the organism, and
the capsule-type drug release apparatus system further comprises receiving means capable of receiving a signal outside the organism, the signal being transmitted by the transmitting portion.
